(19) United States
(12) Patent Application Publication
(10) Pub. No.: US 2022/0051312 A1
(43) Pub. Date: Feb. 17, 2022

(54) Method and System for Choosing Prosthetics Through Cross-Reference of Databases

(71) Applicants: Aldo Laghi, Pinellas Park, FL (US); Alps South Europe, S.R.O., Plzen (CZ)

(72) Inventors: Aldo Laghi, Pinellas Park, FL (US); Mattia Rampazzo, Bologna (IT); Silvia Albertini, Cento (FE) (IT)

(21) Appl. No.: 17/399,270

(22) Filed: Aug. 11, 2021

Related U.S. Application Data
(60) Provisional application No. 63/064,434, filed on Aug. 12, 2020.
(60) Provisional application No. 63/116,175, filed on Nov. 20, 2020.

(51) Int. Cl. G06Q 30/06; A61F 2/50; A61F 2/76

(52) U.S. Cl. CPC G06Q 30/0643; A61F 2/7812; A61F 2/76; A61F 2/5046

(57) ABSTRACT

A method and system of choosing a prosthetic liner using cross-references databases which informs a user using a specified interface. Specifically, the method comprises a user accessing a graphical interface wherein amputee attributes are entered in order of (1) level of amputation, (2) residual limb length, (3) activity level, (4) residual limb shape/volume, and (5) clinical conditions with corresponding attribute options. Alternatively, the chosen attributes are (1) class of amputation, (2) residual limb length, (3) body weight, (4) use of the prosthesis, (5) activity level, and 6) uneven paths for knee joints with corresponding attribute options. Alternatively, the chosen attributes are (1) level of amputation, (2) residual limb length, (3) body weight, (4) use of the prosthesis, (5) activity level, and 6) uneven paths for foot joints with corresponding attribute options. These amputee attributes are then cross-referenced against one another and compared to a second database containing liners that correspond to the amputee attributes selected thereby educating the user, generally a prosthetist, on the appropriate liner style for a particular amputee.

### CROSS-REFERENCE TO RELATED APPLICATIONS

This application claims priority to U.S. Provisional Application No. 63/064,434 titled “Method and System of Choosing Prosthetic Liner Through Cross-Reference of Databases” filed on Aug. 12, 2020, as well as priority to U.S. Provisional Application No. 63/116,175 titled “Method and System of Choosing Prosthetic Liner Through Cross-Reference of Databases” filed on Nov. 20, 2020. The contents of each of the above-listed applications are hereby incorporated by reference herein in their entirety.

### TECHNICAL FIELD

This disclosure relates to a method and system for choosing specific prosthetic articles using an computer-based systems. Specifically, this disclosure relates to a system used with a software as a service platform to aid in the specialized selection of prosthetic devices based on a number of factors associated with amputated limbs.

### BACKGROUND OF THE INVENTION

There are several types of prosthetic articles that are available to amputees. As used herein, the term “article” refers to a covering or device used in concert with a prosthetic device or a prosthetic device itself. Articles such as prosthetic liners are the interface of choice among amputees due to their various beneficial characteristics. Modern liner technology allows amputees to employ a liner as the sole interface between their residual limb (also commonly referred to as a residuum or amputation stump) and the interior of the prosthetic socket as compared to wool/knit socks that require multiple layers. The choice of prosthetic liner types is one that requires knowledge about the effects on an amputee based on their lifestyle. A number of different characteristics can affect the type of prosthetic liner that would best fit an individual's lifestyle.

In the present field there are a wide variety of different prosthetic liners. There are liners made of thermoplastic elastomeric gel, silicone, and urethane as well as liners developed for above and below the knee/elbow amputations. Further, liners may feature a locking mechanism at the closed distal end or may rely on vacuum suspension using high friction seals such as those disclosed in U.S. Publication No. 2015/0142132 to Egilsson et al. This second type of liner is also commonly known as a “cushion liner.” Further, liners can be custom based on the amputee's residual limb or may be of a generic shape that will fit a range of residual limb shapes and sizes.

It is also generally known that liners are constructed so as to have a fabric outer layer. These liners feature an inner layer of gel, silicone, or urethane for contacting and cushioning the amputee's residual limb. The outer fabric layer is primarily to increase wear resistance of the liner and facilitating donning/doffing and insertion of the liner-covered residual limb into the prosthetic socket. Such patented fabric-covered liner products made as locking liners and as cushion liners are available from Alps South, LLC in St. Petersburg, Fla.

When an amputee is in need a of liner, they generally consult with a prosthetist; a healthcare professional specializing in making and fitting artificial limbs (prostheses) for people with disabilities which includes artificial legs and arms for people who have had amputations. These prosthetists receive special education and training in graduate programs and may practice in a variety of setting such as hospitals, rehabilitation centers, private practice, and at industrial health centers.

Previously, when the amputee was in need of a liner, they would consult with the prosthetist who would generally ask questions about the amputees lifestyle. The prosthetist would then recommend a liner based on their limited knowledge of the types of liners available in the market. Even if a prosthetist preferred one company's liners over another, she may not be aware of all liner options that that company has to offer and may even recommend an incorrect liner type if her knowledge of reasons to use a certain liner are out of date. Even if a prosthetist went online for a liner, current systems only list a company's catalogue of liners and the prosthetist will not know which liner will best align with the amputee's lifestyle/attributes.

Liner characteristics that are of importance include, but are not limited to, surface characteristics, modulus of elasticity, thermal conductivity, fabric elasticity, distal constriction, germ adhesion, distal suspension type, and proximal suspension type. Clinical characteristics of the residual limb that are of importance include, but are not limited to, skin health, perspiration, muscular condition, fleshiness of the residual limb, residual limb length, amputation location, neuron exposure, the amputee's activity level, amputee expectations about lifestyle, environment, hygiene, the amputee's manual dexterity, and the amputee's weight.

Likewise, prosthetic knee joints have greatly evolved in the last decades, from a mere passive joint to joints with adjustable response in the load support (stance) phase and/or in a swing phase (flexion/extension) and to active microprocessor controlled joint. The choice of prosthetic knee joint types is one that requires knowledge about the effects on an amputee based on their lifestyle. A number of different characteristics can affect the type of prosthetic knee joints that would best fit an individual's lifestyle and needs.

In the present field there are a wide variety of different prosthetic knee joints. There are prosthetic knee joints with polycentric or uniaxial geometry; with the possibility to adjust the support during the stance phase; or to adjust the response during the flexion and extension of the leg using a different set of technologies; or knees which response is determined by a microprocessor embedded into the unit.

As functional performances and features differs from one prosthetic knee to another, there is also an important variation in cost between different types of prosthetic knees. As typically the prosthetic leg is reimbursed in total or in part by national reimbursement systems or insurances and such reimbursements vary from country to country, the cost of the prosthetic knee is an additional key factor in the selection process

When a transfemoral amputee is in need a of a prosthetic leg, in order to select the more suitable knee joint, they generally consult with a prosthetist; a healthcare professional specializing in making and fitting artificial limbs (prostheses) for people with disabilities which includes artificial legs and arms for people who have had amputations. These prosthetists receive a special education and training in graduate programs and may practice in a variety of setting such as hospitals, rehabilitation centers, private practice, and at industrial health centers.

Previously, when the amputee was in need of a prosthetic leg joint, they would consult with the prosthetist who would generally ask questions about the amputees' lifestyle to select the proper components—including the prosthetic knee. The prosthetist would then recommend a prosthetic knee based on their limited knowledge of the types of knees available in the market. Even if a prosthetist preferred one company's prosthetic knees over another, he may not be aware of all prosthetic knees options that that company has to offer and may even recommend an inappropriate prosthetic knee type if his knowledge of reasons to use a certain knee are out of date.

Knee characteristics that are of importance include, but are not limited to knee geometry, different technologies to provide support during load and stance phase, possibility to lock the knee to provide increased safety, different technologies to allow the knee to flex, different technologies to make possible the extension of the leg, possibility to fit long residual limbs and knee disarticulated amputees, ability to change the response during stance, flexion and/or extension accordingly to the specific characteristics of the amputee, easy initiation of the knee flexion, capability to allow walking at different walking speeds, increase the balance of the amputee on uneven surfaces, cost. Clinical characteristics of the residual limb that are of importance include, but are not limited to, skin health, muscular condition, residual limb length, amputation location, the amputee's activity level, amputee expectations about lifestyle, environment the amputee has to face, the amputee's manual dexterity, and the amputee's weight.

Prosthetic feet have greatly evolved in the last decades, from a wooden rigid foot, to feet with axial compliance and/or with the capability to store and return energy, to feet with a microprocessor-controlled response. The choice of prosthetic foot types is one that requires knowledge about the effects on an amputee based on their lifestyle. A number of different characteristics can affect the type of prosthetic foot that would best fit an individual's lifestyle and needs.

In the present field there are a wide variety of different prosthetic feet. There are prosthetic feet with a rigid ankle and a soft heel; or that allow for plantarflexion and dorsiflexion; or for multiaxial compliance; or that can absorb and return different amounts of energy to facilitate the gait cycle; or feet which response is determined by a microprocessor embedded into the unit.

As functional performances and features differs from one prosthetic foot to another, there is also an important variation in cost between different types of prosthetic feet. As typically the prosthetic leg is reimbursed in total or in part by national reimbursement systems or insurances and such reimbursements vary from country to country, the cost of the prosthetic foot is an additional key factor in the selection process.

When an amputee is in need a of a prosthetic leg, in order to select the more suitable foot, they generally consult with a prosthetist; a healthcare professional specializing in making and fitting artificial limbs (prostheses) for people with disabilities which includes artificial legs and arms for people who have had amputations. These prosthetists receive a special education and training in graduate programs and may practice in a variety of setting such as hospitals, rehabilitation centers, private practice, and at industrial health centers.

Previously, when the amputee was in need of a prosthetic leg, they would consult with the prosthetist who would generally ask questions about the amputees' lifestyle to select the proper components—including the prosthetic foot. The prosthetist would then recommend a prosthetic foot based on their limited knowledge of the types of feet available in the market. Even if a prosthetist preferred one company's prosthetic feet over another, he may not be aware of all prosthetic feet options that that company has to offer and may even recommend an inappropriate prosthetic foot type if his knowledge of reasons to use a certain foot are out of date.

Feet characteristics that are of importance include, but are not limited to, ability to dampen the heel strike, ability to allow for safe contact with the ground, ability to facilitate the foot progression from late stance to push off, ability to adapt to uneven terrains, ability to provide a variable dynamic response accordingly to the characteristics of the gait, weight of the device, fitting height and cost.

Clinical characteristics of the residual limb that are of importance include, but are not limited to skin health, muscular condition, residual limb length, amputation location, the amputee's activity level, amputee expectations about lifestyle, environment the amputee has to face, the amputee's manual dexterity, and the amputee's weight.

Thus, it would be an improvement in the art to provide a system that could aid in the education of prosthetists about prosthetic articles that are available based on the characteristics and limitations of an amputee's lifestyle.

The present inventions comprises a system comprising multiple databases containing article characteristics and clinical characteristics in combination with a user interface that allows a prosthetist to step through each of the requirements an amputee might need in order to determine which liner is the best fit.

Therefore, it is an object of this invention to provide an improvement which overcomes the aforementioned inadequacies of the prior art and provides an improvement which is a significant contribution to the advancement of the prosthetic article selection method art.

A further object of the present invention is to provide a user interface which allows a prosthetist to step through various attributes that inform the proper liner fit.

A further object of the present invention is to interface with an external database which stores various attributes in combination with liner types further in communication with the user.

A further object of the present invention is to provide a method of selecting a prosthetic liner that educates the user, whether the user is a prosthetist or an amputee.

A further object of the present invention is to quickly and efficiently make a determination concerning liner choice between at least 180 different combinations.

A further object of the present invention is to provide a “living tool” to help both the prosthetist and the amputee to optimize the selection of the most appropriate liner which would include liner updates, new products, new functionalities, new clinical conditions, geographical location, local reimbursements, climatic conditions, and seasonal factors, among other options.

A further object of the present invention is to provide a system that does not require training.

A further object of the present invention is to provide a user interface which allows a prosthetist to step through various attributes that inform the proper prosthetic knee joint fit.

A further object of the present invention is to interface with an external database which stores various attributes in combination with prosthetic types further in communication with the user.

A further object of the present invention is to provide a method of selecting a prosthetic knee joint that educates the user, whether the user is a prosthetist or an amputee.

A further object of the present invention is to provide a classification of the large variety of knee joints available on the market, grouping them into a limited number of knee categories and to educate the user, whether the user is a prosthetist or an amputee, which categories fit the best.

A further object of the present invention is to quickly and efficiently make a determination concerning prosthetic knee choice between at least 900 different combinations.

A further object of the present invention is to allow an underneath database, with advanced search options, that allow to browse through a wide variety of prosthetic knee joints from the main manufacturers of prosthetic devices.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

For the purpose of summarizing this invention, this invention comprises a method and system of choosing a prosthetic liner using cross-references databases which informs a user using a specified interface. Specifically, the method comprises a user accessing a graphical interface wherein amputee attributes are entered in order of (1) level of amputation, (2) residual limb length, (3) activity level, (4) residual limb shape/volume, and (5) clinical conditions with corresponding attribute options. Alternatively, the chosen attributes are (1) class of amputation, (2) residual limb length, (3) body weight, (4) use of the prosthesis, (5) activity level, and 6) uneven paths for knee joints with corresponding attribute options. Alternatively, the chosen attributes are (1) level of amputation, (2) residual limb length, (3) body weight, (4) use of the prosthesis, (5) activity level, and 6) uneven paths for foot joints with corresponding attribute options. These amputee attributes are then cross-referenced against one another and compared to a second database containing liners that correspond to the amputee attributes selected thereby educating the user, generally a prosthetist, on the appropriate liner style for a particular amputee.

Embodiments of the present invention are herein described by way of example and are directed to a system for selecting prosthetic articles using a software as a service platform. The aforementioned state of the art of prosthetic article selection methods shows the need for improvements, specifically in the ability of rapid provision of liner style when comparing against certain amputee conditions.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIGS. 1-5 depict the user interface of a first embodiment of the present invention;

FIG. 6 provides a view of the system of a first embodiment of the present invention;

FIGS. 7-12 depict the user interface of a second embodiment of the present invention;

FIG. 13 provides a view of the system of a second embodiment of the present invention; and

FIGS. 14-20 depict the user interface of a third embodiment of the present invention.

Similar reference numerals refer to similar parts throughout the several views of the drawings.

### DETAILED DESCRIPTION OF THE DRAWINGS

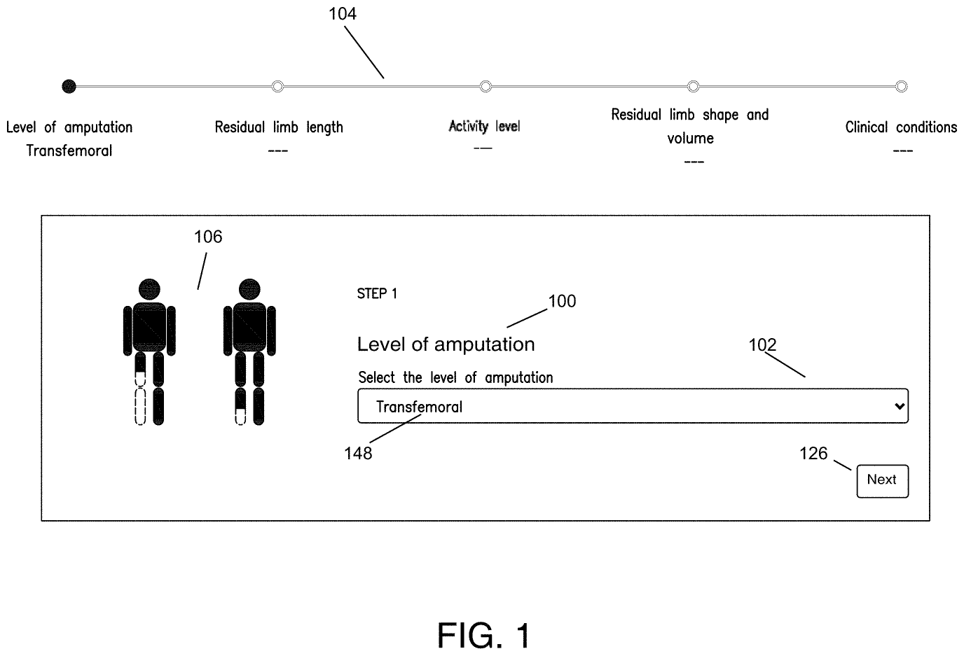

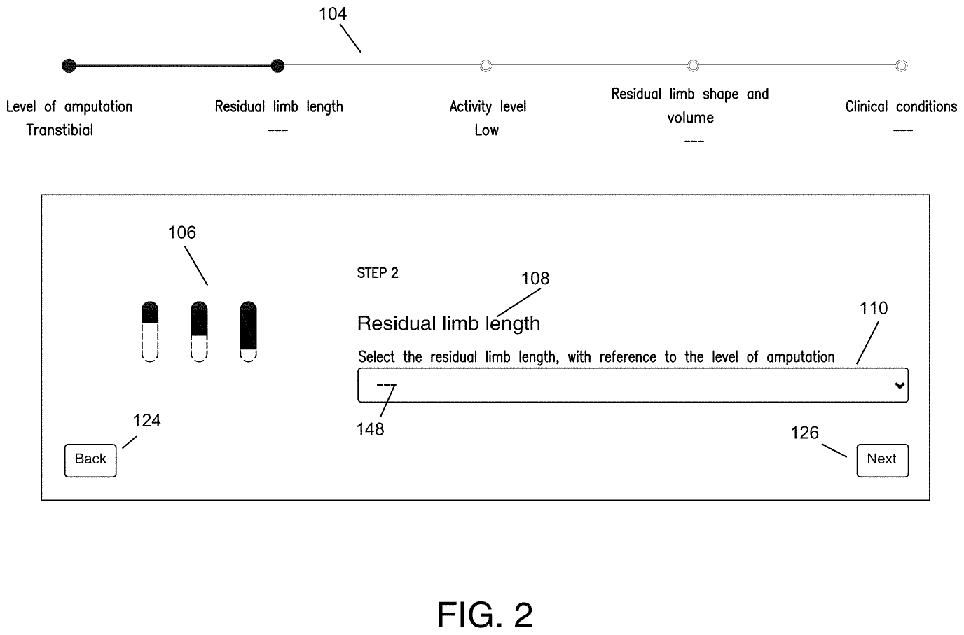

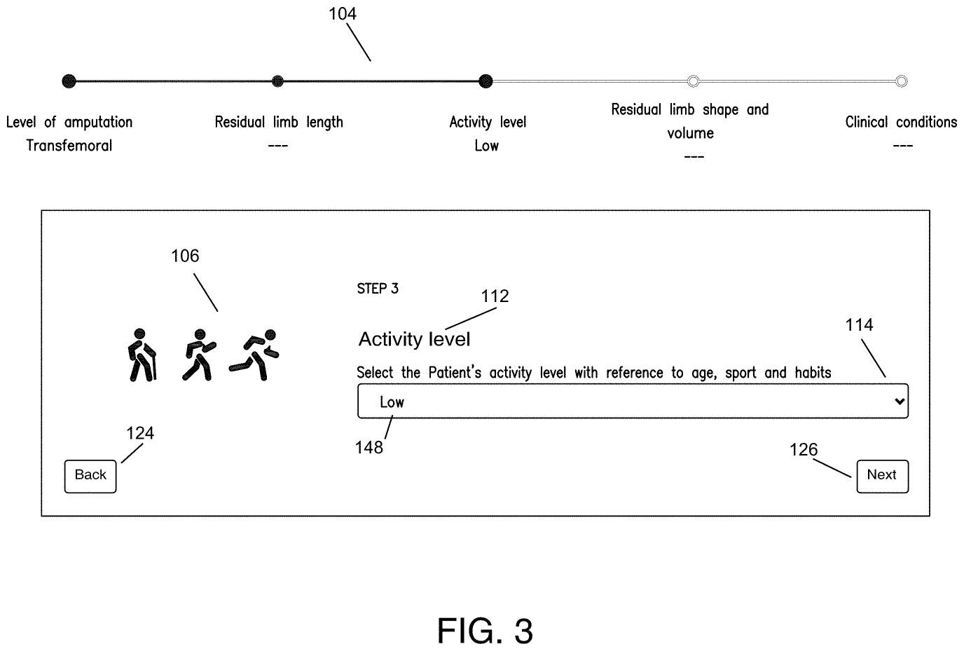

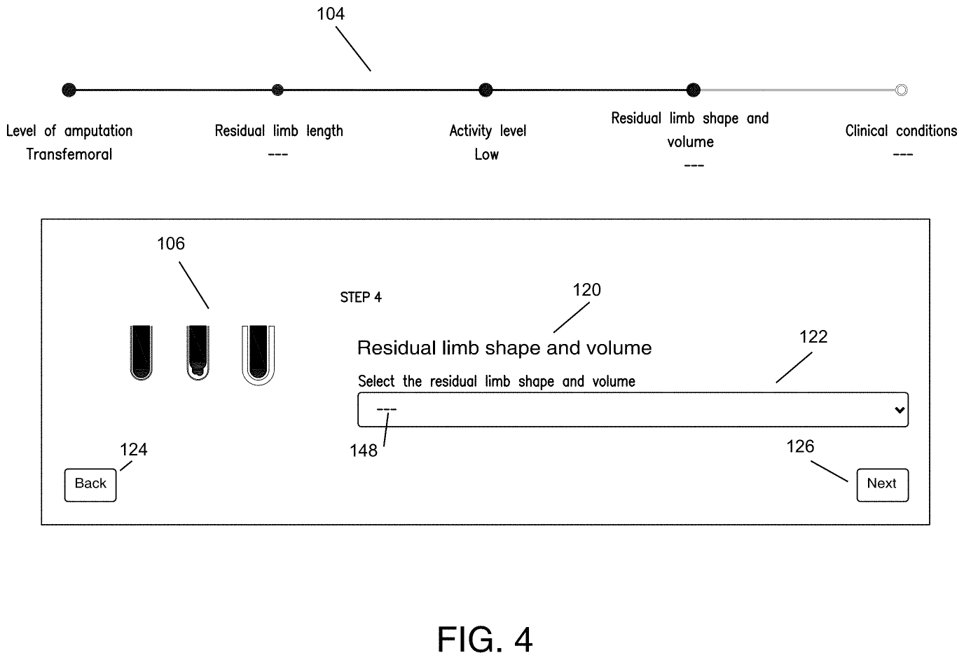

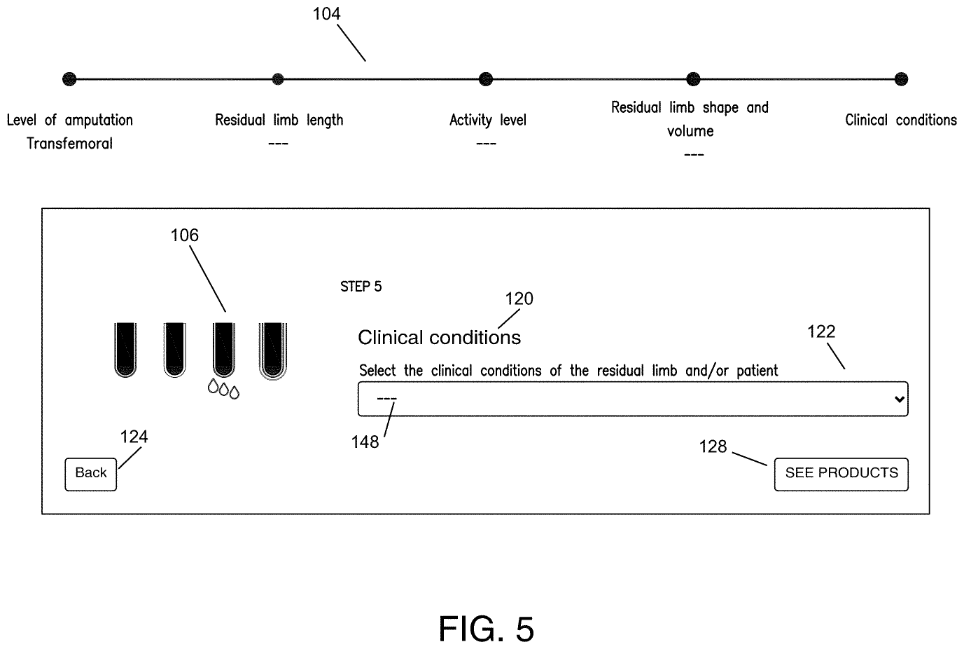

The present invention relates to a method and system for choosing a prosthetic liner through cross-referencing databases. The present invention begins by determining the level of amputation an amputee has; transfemoral or transtibial. As shown in FIG. 1, the first amputee attribute 100 having a plurality of attribute options 148, in this case level of amputation which can be transfemoral or transtibial, is recorded using a first decision box 102 which stores the recorded first amputee attribute 100 in a first database. Each step of the process can be followed along by use of a tracking bar 104 and further comprises a visual depiction 106 of the amputee attribute being chosen. After the first amputee attribute 100 is made using the first decision box 102, the user moves onto a determination of residual limb length. As shown in FIG. 2, a residual limb can be short, medium, or long. This second amputee attribute 108 is recorded using a second decision box 110 which is then also stored in the first database. As shown in FIG. 3, the next amputee attribute determination is activity level of the amputee which can be high, medium, or low. This third amputee attribute 112 is recorded using a third decision box 114 which is also then stored in the first database 132. As shown in FIG. 4, the next amputee attribute determination is the shape and volume of the residual limb which can either be regular, bony, or redundant/fleshy. This fourth amputee attribute 116 is recorded using a fourth decision box 118 which is also then stored in the first database. Finally, as shown in FIG. 5, the fifth amputee attribute 120 determination is other clinical conditions. The user has a choice between (1) normal conditions, (2) diabetic or sensitive skin, (3) abundant perspiration, or (4) daily volume fluctuations. The user makes a choice and then the fifth amputee attribute 120 is recorded using a fifth decision box 122 which is also then stored in the first database. During each step of the process, the user has the option of choosing a “back” choice 124 to go back a step or choosing a “next” choice 126 to move onto the next step with the exception of the first amputee attribute choice step (no “back” choice 124) and the fifth amputee attribute choice step which instead has a submission option 128.

It is important to note that the number of amputee attributes may vary in the system and more or less than five attributes can be implemented using this system. Similarly, the attributes described herein may have modified options depending on the amputee using the system which can be easily added by modifying the options presented in the user interface 136. For example, the clinical condition associated with the fifth amputee attribute may include other conditions that amputees or prosthetists may suggest.

Once the amputee attributes are chosen and recorded in the first database 132, a check is made to a second database 134 housing every combination of amputee attribute associated with choosing a prosthetic liner, as described above. The second database 134 is preferably encrypted to maintain any trade secret determinations for liner choice and is preferably kept separate from the first database. After the last amputee attribute is stored in the first database 132, the system then maps each amputee attribute to associated columns 152, with the first amputee attribute 100 mapping to a first column 154, the second amputee attribute 108 mapping to a second column 156, the third amputee attribute mapping to a third column 158, the fourth amputee attribute mapping to a fourth column 160, and the fifth amputee attribute mapping to a fifth column 162. Each of the attribute options 148 are then matched in associated rows. Once the system finds a match for each attribute option 148, a check is then made to at least one additional column 164 containing the information associated with a specific type of liner and the liner information is returned to the user. Preferably, in situations where more than one liner may be used for specific amputee attributes, more than one column of data may be returned.

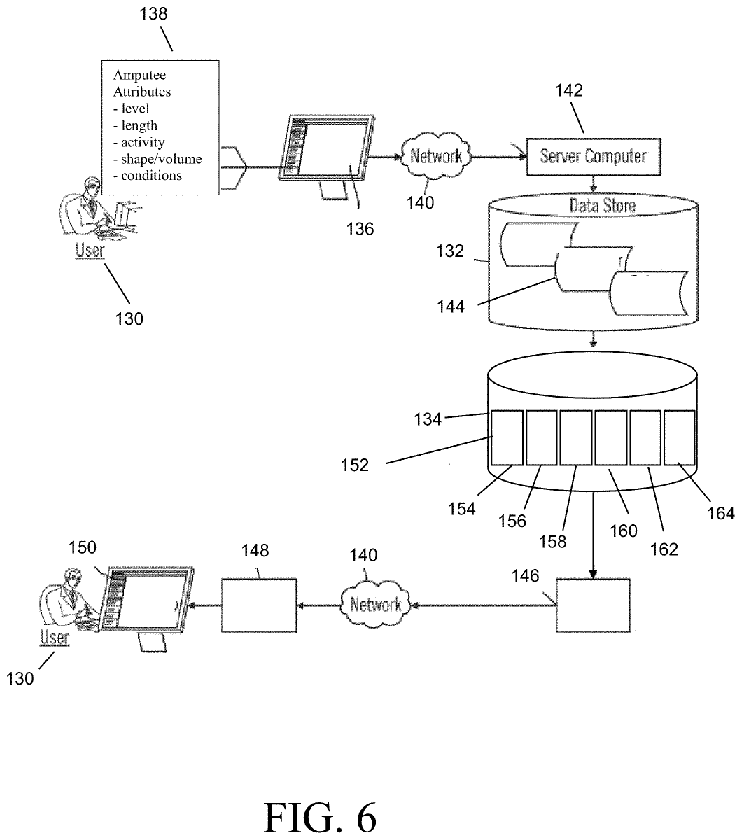

As can be seen in FIG. 6, the system of the present invention comprises a user 130, the first database 132, the second database 134, and a user interface 136. Once the amputee attributes 138 are input into the user interface 136, the information is sent over a network 140 to a server computer 142 which stores the first database 132. After all of the amputee attributes 138 are stored in the first database 132, the attribute grouping 144 is then sent to the second database 134 for mapping as described in the paragraph above. When the desired liner type is determined, the second database 134 sends out a decision choice 146 over the network 140 back to the user's local server 148 which provides a display of the decision choice 146 to the user 130.

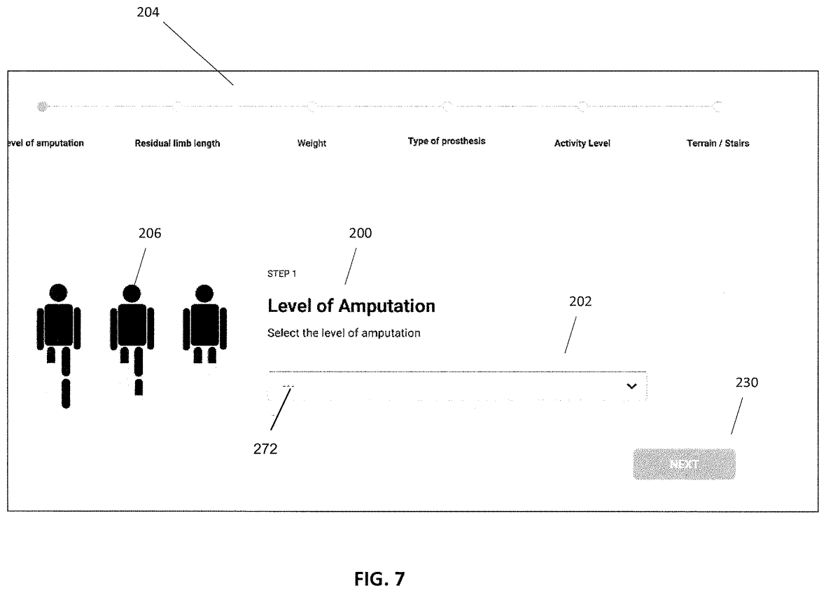

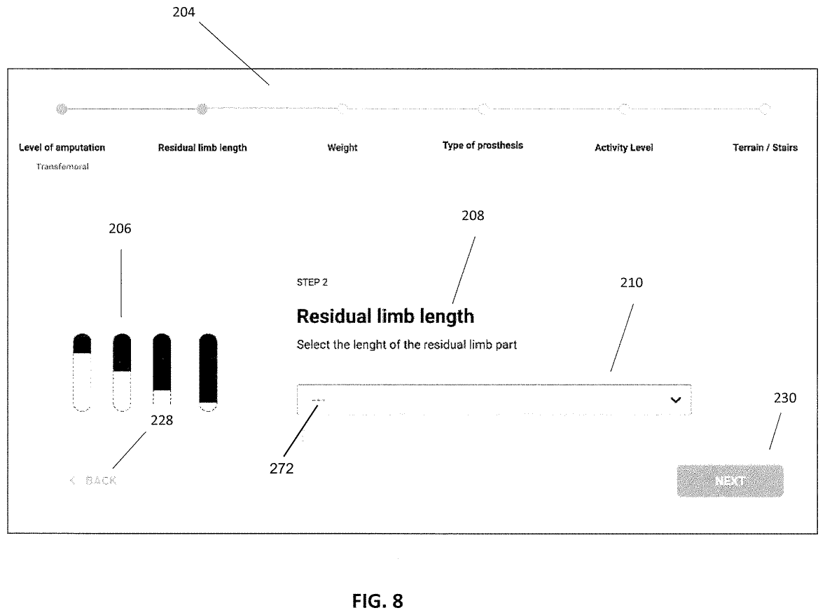

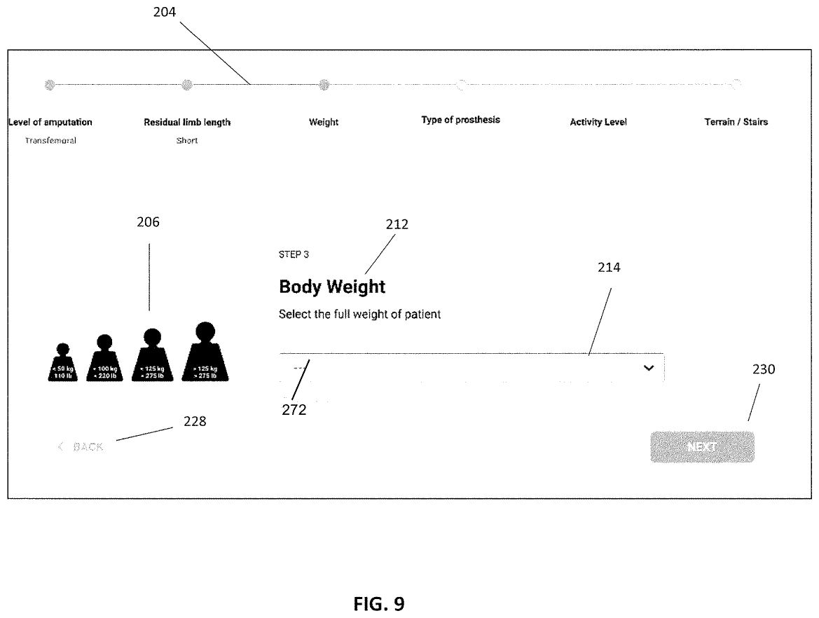

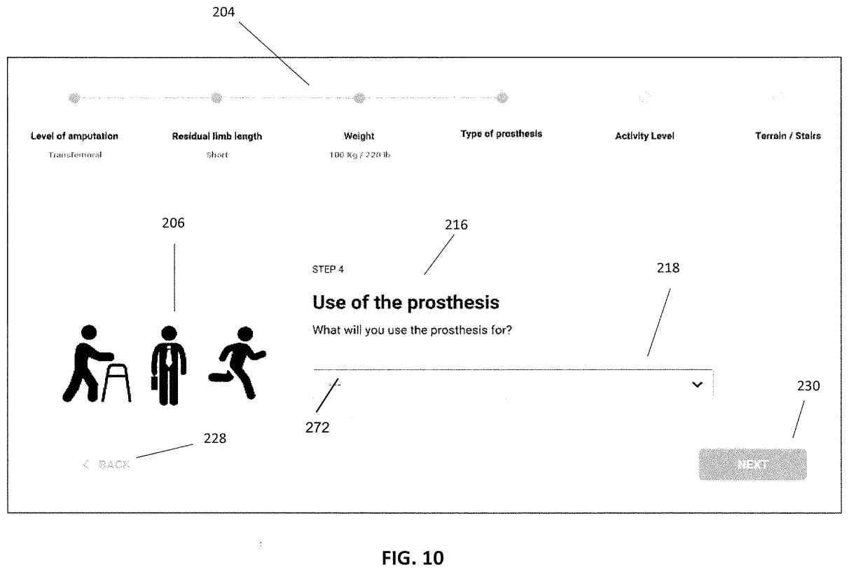

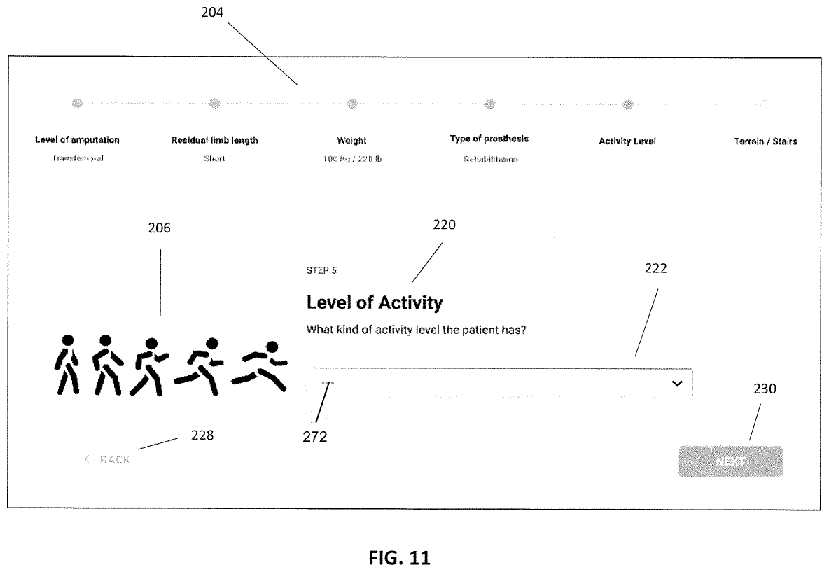

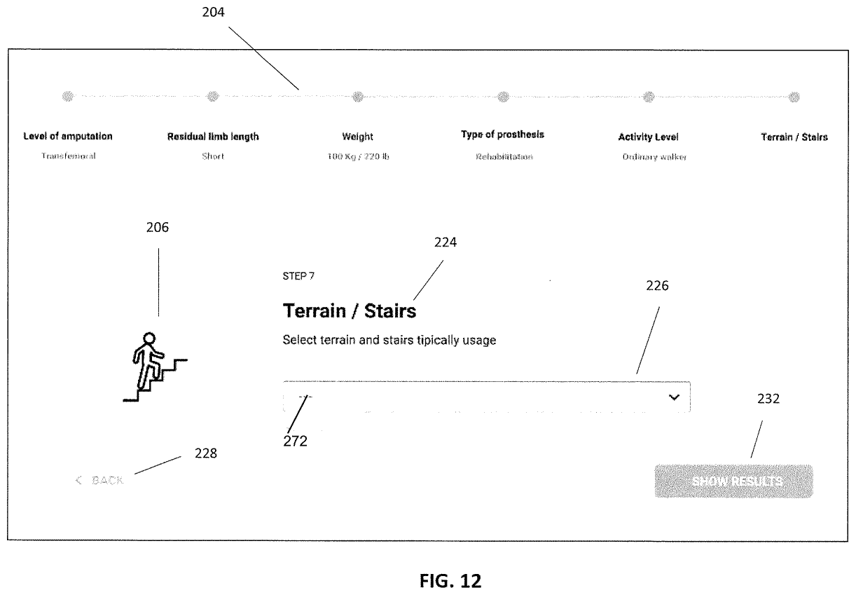

A second embodiment of the present invention is shown in FIGS. 7-12. The present invention begins by determining the level of amputation an amputee has; transfemoral or bilateral transtibial/transfemoral or bilateral transfemoral/transfemoral. As shown in FIG. 7, the first amputee attribute 200 comprising a plurality of attribute options 272, in this case class of amputation which can be transfemoral or bilateral transtibial/transfemoral or bilateral transfemoral/transfemoral, is recorded using a first decision box 202 which stores the recorded first amputee attribute 200 in a first database. Each step of the process can be followed along by use of a tracking bar 204 and further comprises a visual depiction 206 of the amputee attribute being chosen. After the first amputee attribute 200 is made using the first decision box 202, the user moves onto a determination of residual limb length. As shown in FIG. 8, a residual limb can be short, medium, long or knee disarticulation. This second amputee attribute 208 is recorded using a second decision box 210 which is then also stored in the first database. As shown in FIG. 9, the next amputee attribute determination is the body weight of the amputee. The user has a choice between (1)<50 Kg/110 lb, (2)<100 kg/220 lb, (3)<125 Kg/275 lb, (4) above 125 kg/275 lb. This third amputee attribute 212 is recorded using a third decision box 214 which is also then stored in the first database. As shown in FIG. 10, the next amputee attribute determination is the use of the prosthesis which can either be (1) initial prosthesis (2) everyday life or (3) sport only. This fourth amputee attribute 216 is recorded using a fourth decision box 218 which is also then stored in the first database. As shown in FIG. 11, the fifth amputee attribute 220 determination is the level of activity of the amputee. The user has a choice between indoor walker (K1), limited outdoor walker (K2), ordinary walker (K3), dynamic walker (K3+) or very active user (K4). The user makes a choice and then the fifth amputee attribute 220 is recorded using a fifth decision box 222 which is also then stored in the first database. Finally, as shown in FIG. 12, the sixth amputee attribute is related to his lifestyle, specifically if he has to deal with uneven paths. The user has a choice between (1) YES and (2) NO. This sixth amputee attribute 224 is recorded using a fourth decision box 226 which is also then stored in the first database During each step of the process, the user has the option of choosing a “back” choice 228 to go back a step or choosing a “next” choice 230 to move onto the next step with the exception of the first amputee attribute choice step (no “back” choice 228) and the sixth amputee attribute choice step which instead has a submission option 232.

Once the amputee attributes are chosen and recorded in the first database 236, a check is made to a second database 238 housing every combination of amputee attribute associated with choosing a prosthetic knee joints, as described above. The second database 238 is preferably encrypted to maintain any trade secret determinations for knee choice and is preferably kept separate from the first database. After the last amputee attribute is stored in the first database 236, the system then maps each amputee attribute to associated columns 256, with the first amputee attribute 200 mapping to a first column 258, the second amputee attribute 208 mapping to a second column 260, the third amputee attribute mapping to a third column 262, the fourth amputee attribute mapping to a fourth column 264, the fifth amputee attribute mapping to a fifth column 266 and the sixth amputee attribute mapping to a sixth column 268. Each of the attribute options 272 are then matched in associated rows. Once the system finds a match for each attribute option 272, a check is then made to at least one additional column 270 containing the information associated with a specific type of knee joints and the prosthetic knee joint information is returned to the user. Preferably, in situations where more than one knee joint may be used for specific amputee attributes, more than one column of data may be returned.

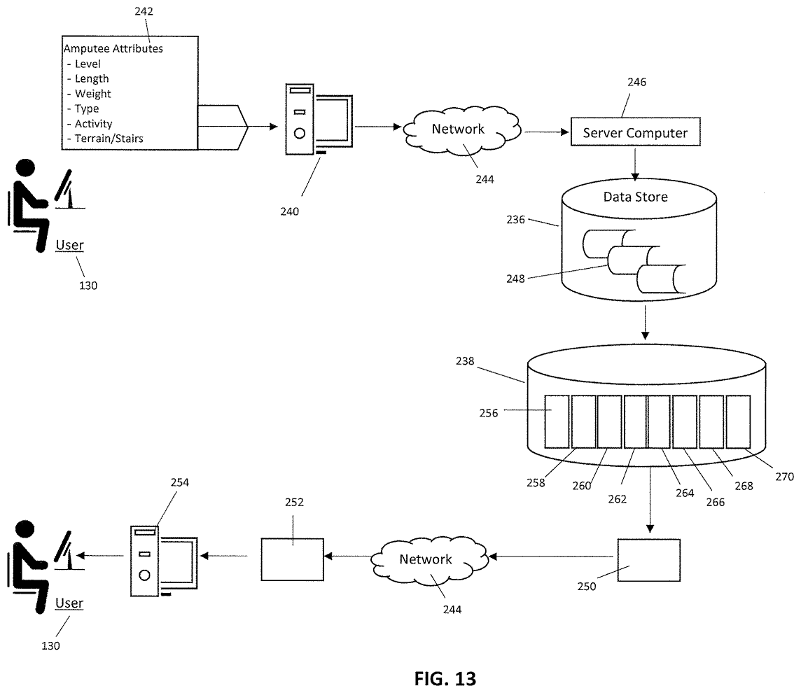

As can be seen in FIG. 13, the system of the present invention comprises a user 130, the first database 236, the second database 238, and a user interface 240. Once the amputee attributes 242 are input into the user interface 240, the information is sent over a network 244 to a server computer 246 which stores the first database 236. After all of the amputee attributes 242 are stored in the first database 236, the attribute grouping 248 is then sent to the second database 238 for mapping as described in the paragraph above. When the desired knee joint type is determined, the second database 238 sends out a decision choice 250 over the network 244 back to the user's local server 252 which provides a display of the decision choice 250 to the user 130.

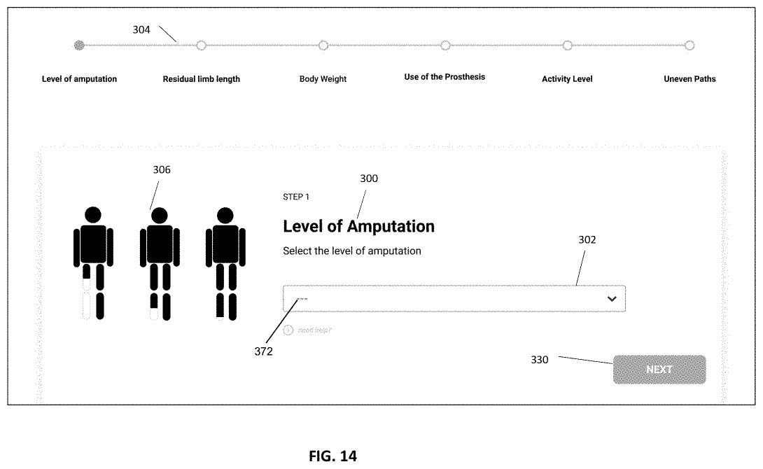

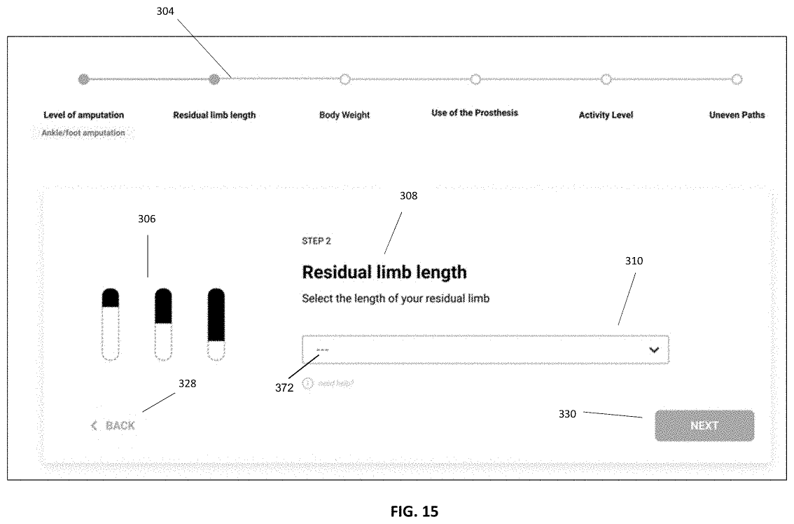

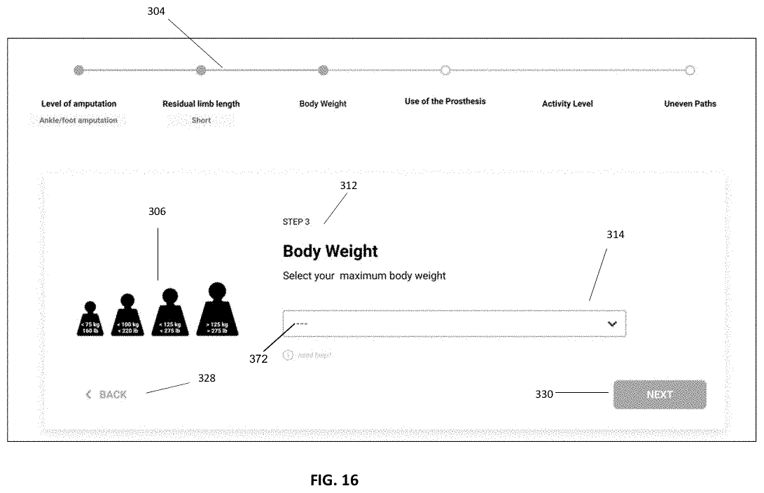

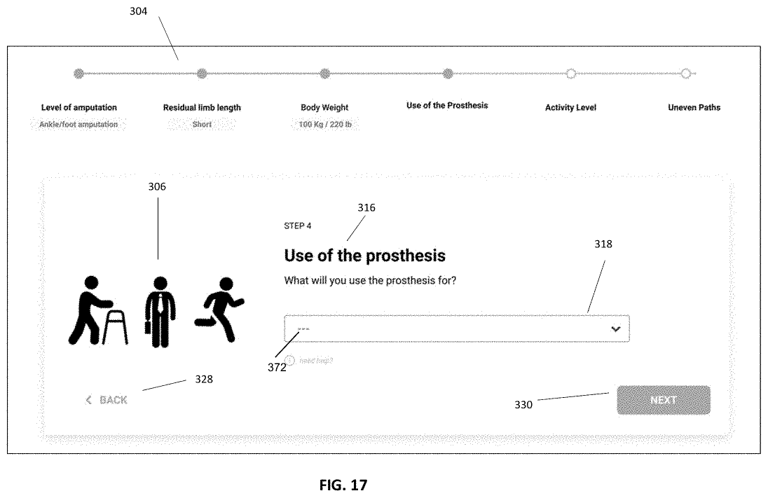

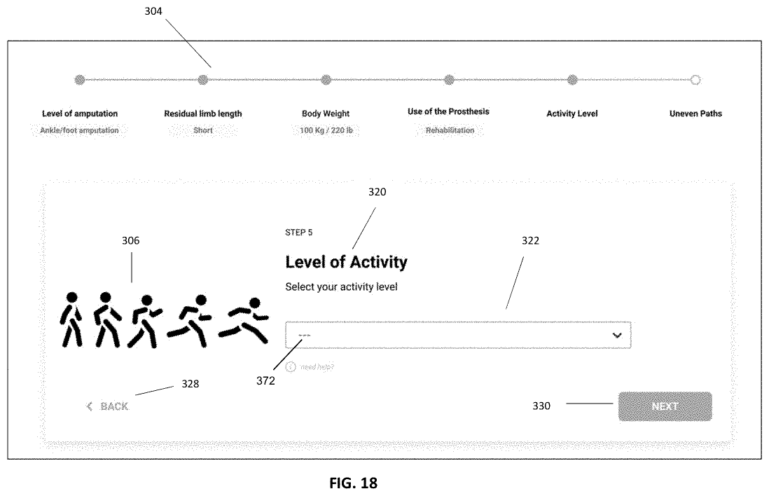

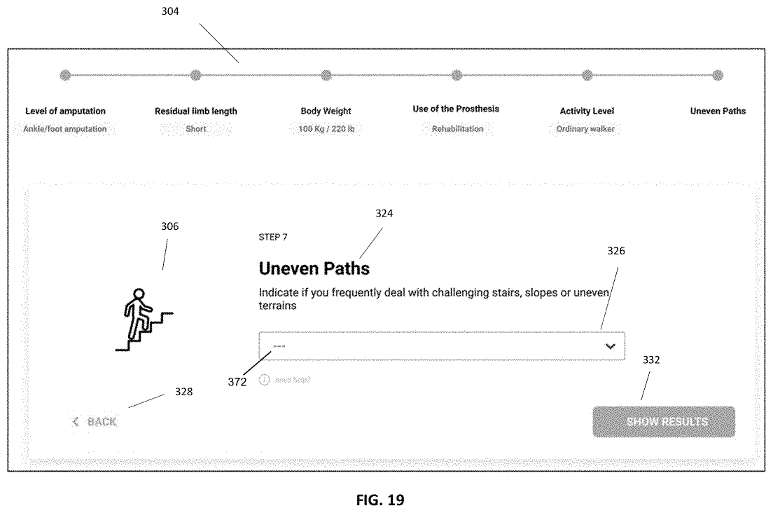

A third embodiment of the present invention is shown in FIGS. 14-20. The present invention begins by determining the level of amputation an amputee has: transfemoral or transtibial or ankle/foot amputation. As shown in FIG. 14, the first amputee attribute 300 comprising a plurality of attribute options 372, in this case class of amputation which can be transfemoral or transtibial or ankle/foot amputation, is recorded using a first decision box 302 which stores the recorded first amputee attribute 300 in a first database. Each step of the process can be followed along by use of a tracking bar 304 and further comprises a visual depiction 306 of the amputee attribute being chosen. After the first amputee attribute 300 is made using the first decision box 302, the user moves onto a determination of residual limb length. As shown in FIG. 15, a residual limb can be short, medium or long. This second amputee attribute 308 is recorded using a second decision box 310 which is then also stored in the first database. As shown in FIG. 16, the next amputee attribute determination is the body weight of the amputee. The user has a choice between (1)<75 Kg/165 lb, (2)<100 kg/220 lb, (3)<125 Kg/275 lb, (4) above 125 kg/275 lb. This third amputee attribute 312 is recorded using a third decision box 314 which is also then stored in the first database. As shown in FIG. 17, the next amputee attribute determination is the use of the prosthesis which can either be (1) initial prosthesis (2) everyday life or (3) sport only. This fourth amputee attribute 316 is recorded using a fourth decision box 318 which is also then stored in the first database. As shown in FIG. 18, the fifth amputee attribute 320 determination is the level of activity of the amputee. The user has a choice between indoor walker (K1), limited outdoor walker (K2), ordinary walker (K3), dynamic walker (K3+) or very active user (K4). The user makes a choice and then the fifth amputee attribute 320 is recorded using a fifth decision box 322 which is also then stored in the first database. Finally, as shown in FIG. 19, the sixth amputee attribute is related to his lifestyle, specifically if he has to deal with uneven paths. The user has a choice between (1) YES and (2) NO. This sixth amputee attribute 324 is recorded using a sixth decision box 326 which is also then stored in the first database During each step of the process, the user has the option of choosing a “back” choice 328 to go back a step or choosing a “next” choice 330 to move onto the next step with the exception of the first amputee attribute choice step (no “back” choice 328) and the sixth amputee attribute choice step which instead has a submission option 332.

Once the amputee attributes are chosen and recorded in the first database 336, a check is made to a second database 338 housing every combination of amputee attribute associated with choosing a prosthetic foot, as described above. The second database 338 is preferably encrypted to maintain any trade secret determinations for foot choice and is preferably kept separate from the first database. After the sixth amputee attribute 324 is stored in the first database 336, the system then maps each amputee attribute to associated columns 356, with the first amputee attribute 300 mapping to a first column 358, the second amputee attribute 308 mapping to a second column 360, the third amputee attribute mapping to a third column 362, the fourth amputee attribute mapping to a fourth column 364, the fifth amputee attribute mapping to a fifth column 366 and the sixth amputee attribute mapping to a sixth column 368. Each of the attribute options 372 are then matched in associated rows. Once the system finds a match for each attribute option 372, a check is then made to at least one additional column 370 containing the information associated with a specific type of foot and the prosthetic foot information is returned to the user. Preferably, in situations where more than one foot may be used for specific amputee attributes, more than one column of data may be returned.

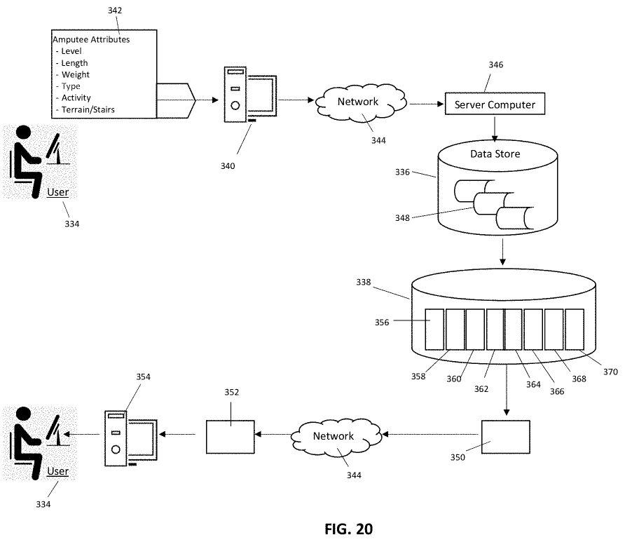

As can be seen in FIG. 20, the system of the present invention comprises a user 334, the first database 336, the second database 338, and a user interface 340. Once the amputee attributes 342 are input into the user interface 340, the information is sent over a network 344 to a server computer 346 which stores the first database 336. After all of the amputee attributes 342 are stored in the first database 336, the attribute grouping 348 is then sent to the second database 338 for mapping as described in the paragraph above. When the desired foot type is determined, the second database 338 sends out a decision choice 350 over the network 344 back to the user's local server 352 which provides a display of the decision choice 350 to the user 334.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

What is claimed is:

1. A method of choosing a prosthetic article comprising:
(A) accessing a graphical interface on a user computer, the graphical interface further comprising a tracking bar and a visual depiction;
(B) showing at least one amputee attribute having a plurality of attribute options on the graphical interface wherein the visual depiction depicts the amputee attribute shown on the graphical interface;
(C) recording a user's choice of attribute option using a decision box;
(D) storing each of the plurality of amputee options in a first database via secure connection over a network to a server computer;
(E) repeating steps (B)-(D) for at least a total of three amputee options creating an attribute grouping;
(F) comparing the attribute grouping to an encrypted second database separated from the first database structurally and comprising columns mapped to corresponding amputee attributes and rows mapped to corresponding attribute options wherein, once a match is made between the attribute grouping and the corresponding amputee attributes and attribute options stored in the second database, a reference is made to a recommendation column comprising a plurality of decision choices in a plurality of rows;
(G) sending a decision choice over the network to the user computer; and
(H) displaying the decision choice on the user computer.

2. The method of choosing a prosthetic article of claim 1 wherein the amputee attribute is chosen from the group consisting of (1) level of amputation, (2) residual limb length, (3) activity level, (4) residual limb shape/volume, and (5) clinical conditions.

3. The method of choosing a prosthetic article of claim 1 wherein the amputee attribute is chosen from the group consisting of (1) class of amputation, (2) residual limb length, (3) body weight, (4) use of the prosthesis, (5) activity level, and 6) uneven paths for knee joints.

4. The method of choosing a prosthetic article of claim 1 wherein the amputee attribute is chosen from the group consisting of (1) level of amputation, (2) residual limb length, (3) body weight, (4) use of the prosthesis, (5) activity level, and 6) uneven paths for foot joints.

5. The method of choosing a prosthetic article of claim 1 wherein the method repeats steps (B)-(D) for at least five amputee options to create the attribute grouping.

6. The method of choosing a prosthetic article of claim 1 wherein a second decision choice is sent to over the network to the user computer if more than one decision choice matches the attribute grouping.

7. A system for choosing a prosthetic article comprising:
a user;
a user interface to input user choices and receive a decision choice connected to a local server;
a first database stored on a server computer to store a plurality of attribute options based on a plurality of amputee attributes creating an attribute grouping;
an encrypted second database stored on the server computer but segregated from the first database comprising a plurality of columns mapped to amputee attributes and rows mapped to attribute options and a recommendation column comprising a plurality of decision choices;
a network allowing the user to send the attribute grouping to the encrypted second database and to send the decision choice back to the user and then displayed on the user interface on the local server.

8. The system of claim 7 wherein the amputee attribute is chosen from the group consisting of (1) level of amputation, (2) residual limb length, (3) activity level, (4) residual limb shape/volume, and (5) clinical conditions.

9. The system of claim 7 wherein the amputee attribute is chosen from the group consisting of (1) class of amputation, (2) residual limb length, (3) body weight, (4) use of the prosthesis, (5) activity level, and 6) uneven paths for knee joints.

10. The system of claim 7 wherein the amputee attribute is chosen from the group consisting of (1) level of amputation, (2) residual limb length, (3) body weight, (4) use of the prosthesis, (5) activity level, and 6) uneven paths for foot joints.

11. The system of claim 7 wherein a plurality of decision choices are sent over the network to the user on the local server if more than one decision choice matches the attribute grouping.
